(19) United States
(12) Patent Application Publication
(10) Pub. No.: US 2008/0234836 A1
(43) Pub. Date: Sep. 25, 2008

(54) Socket preform/adapter combination for prosthetic device and method of manufacture

(76) Inventor: Douglas Taylor, Indianapolis, IN (US)

Correspondence Address: Daniel L. Boots, Bingham McHale LLP, 2700 Market Tower, 10 West Market Street, Indianapolis, IN, 46204-4900, US

(21) Appl. No.: 10/873,597

(22) Filed: Jun. 21, 2004

(51) Int. Cl. A61F 2/80; B29C 70/42

(52) U.S. Cl. 623/33; 623/901; 264/257

(57) ABSTRACT

A socket for connecting a prosthetic limb to the residual limb of a amputee, including an adapter having at least one resin port extending therethrough and a generally conical composite material extending from the adapter. The composite material further includes a resin matrix and a multilayer triaxially woven preform embedded in the matrix. The preform is woven directly onto a positive mold of the residual limb. The adapter is positioned generally at the apex of the generally conical composite material. The adapter protrudes through the composite material.

### CROSS REFERENCE TO RELATED APPLICATIONS

This application claims priority to U.S. provisional patent application Ser. No. 60/354,277, filed Oct. 25, 2001; and also claims priority to PCT application No. PCT/US02/34050, filed Oct. 24, 2002.

### TECHNICAL FIELD OF THE INVENTION

The present invention pertains to prosthetic devices and more particularly pertains to a socket preform/adapter combination and method of manufacture thereof.

### BACKGROUND OF THE INVENTION

Every endoskeletal type prosthesis has a residual limb interface, or socket. The socket includes an adapter to which the prosthetic limb is attached. The socket transfers the stresses of walking and lifting from the patient to the other components of the prosthesis. Sockets are custom measured and fabricated by hand lay-up preforming and hand-controlled, vacuum-assisted resin injection. A socket preform is typically assembled by cutting combinations of woven, knitted and braided fiber textiles and tying and/or tackifying (adhesive tacking) the textile over a positive model of the socket. A single or two-stage lay-up resin injection process, which includes attachment of the adapter, is common. Resin injection is performed by hand pressure and stringing to force resin into the lay-up, and is commonly advanced through the fiber preform by vacuum and a stringing process.

Present adapters are designed to fit into less than desirable composites. Adapters come in three general forms: a flat plate with standardized metric four hole square pattern; a three winged threaded hole with clamp; and an inverted tetrahedron with a dome and four wings. Each design includes large “wings” to prevent the adapter from being torn out of the socket during service. It is possible, however, to use the isotropic (equal in all directions) physical properties strength of metals in conjunction with good composites to decrease socket weight while increasing effective socket strength.

Several factors affect the performance of fiber preform composite technology. The strength, number and orientation of the fibers are important. Fibers have to be aligned so the axis of stress is arranged down the length of the fiber. Fibers should also have a small diameter and be tightly packed together. Fibers should be capable of mechanically and chemically bonding to the resin as well, and the resin should be characterized by a coefficient of expansion and/or elongation greater than that of the fiber so that stress is transferred to the fiber. Socket fiber volume of between about 50-70% is also preferred.

In use, socket loading patterns and forces are constantly changing, both internally and externally. Individual gait cycles further complicate socket performance and design. Since forces inevitably stray from the fiber plane and despite current fiber and resin composition and methods of manufacture, sockets sometimes eventually give way and fail.

Most shortcomings of current materials and methods can be readily identified. Knitted cloths from nylon and fiberglass are generally unsuitable for use in high performance composites. Their fiber orientation is looping, their fiber content is low and adhesion between fiber and resin is poor. Composites made with such materials tend to be weak in tensile strength.

Carbon fiber braids are also widely used in composites. Unfortunately, these filamentary materials are manufactured in constant diameter tubings that result in both tensile jamming of the fiber bundles and reduced resin permeability as the diameter of the mold decreases past the braid minimum diameter limit. This decreases resin flow and increases wet-out time, thus trapping excess resin and weakening the composite in that area. In contrast, where the diameter of the mold increases, permeability increases so that fiber coverage is inadequate. This also reduces the effective overall strength of the resultant form due to localized thinning of the composite.

Some prosthetic sockets may vary in diameter several inches in a short distance. Fortunately, the smallest diameter is usually at the socket adapter where forces are perpendicular to the fiber plane. Increased thickness is required in this region to handle the off-axis stress. If the original diameter of the braid is properly selected coverage can be good for the large diameter of the socket and much thicker at the adapter where it is needed. However, this still leaves questionable fiber orientations in several areas of the socket and inefficient use of composite properties, such as its superior strength along the fiber axis.

Additionally, the primary axis of stress for all prosthetic devices is in the axial plane of the prosthesis, which means that fiber orientation is at an angle of between about 15 to 85 degrees relative to the plane. The stresses in the axial plane are therefore primarily accommodated by the resin matrix, and strength in the axial plane is up to 70% weaker than it is along the fiber axis as a result. Due to the angulation of the force vectors during ambulation, most of the other areas of the socket experience out-of-plane forces at some time as well.

Further, the perform-adapter interface typically includes unevenly cut and folded fiber portions unevenly jammed around the adapter and held in place by hardened resin. Resultantly, the adapter may be poorly positioned to uniformly distribute the forces of ambulation. Moreover, the interface may include weak spots arising from localized concentrations of poorly wetted fibers, folded and bent fibers, and localized concentrations of resin.

There is therefore a need for a fiber-adapter interface characterized by an even distribution of uniformly wetted/wettable fibers. The present invention addresses this need.

### SUMMARY OF THE INVENTION

The present invention relates to a method and apparatus for producing a socket for a prosthetic wearer, and to the socket itself. The socket comprises a woven fiber/resin matrix composite material formed over a positive mold of the wearer's residual limb.

In one preferred embodiment, the present invention relates to a method for making a socket to be worn over a residual limb for connection of a prosthetic limb thereto, including the steps making a positive mold of the residual limb; weaving a layered fibrous preform; connecting the preform to an adapter; applying the preform over the mold; positioning the adapter as desired relative to the positive mold; form-fitting the preform to the shape of the mold such that the preform fits tightly over the mold with substantially no space therebetween; injecting resin through the adapter onto the preform; substantially evenly permeating the preform with resin; curing the resin to form a fibrous preform/resin matrix composite socket; and removing the socket from the positive mold. The at least one layer of the preform includes criss-crossing fibers oriented in at least two axial directions.

In another preferred embodiment, the present invention relates to a socket to be worn over a residual limb for connection of a prosthetic limb thereto, including an adapter having at least one resin port formed therethrough and a composite shell having an inner surface and an outer surface and connected to the adapter. The composite shell extends generally cylindrically away from the adapter. The inner surface of the socket is custom-molded to conform to the contours of the residual limb of a desired wearer. The adapter protrudes through the outer surface of the composite shell. The composite shell further includes a cured resin matrix and a woven fiber preform embedded in the resin matrix.

In still another preferred embodiment, the present invention relates to a jig for producing a composite residual limb socket, including a first substantially L-shaped hollow tubular member having a first first end and a first second end; a second substantially L-shaped hollow tubular member having a second first end and a second second end; a third substantially L-shaped hollow tubular member having a third first end and a third second end; a fourth hollow tubular member having a fourth first end and a fourth second end; a fifth hollow tubular member having a fifth first end and a fifth second end; a resin vial connected in pneumatic communication with the fourth second end; a vacuum port connected in pneumatic communication with the fifth first end; a first vacuum coupling connected in pneumatic communication with the vacuum port; a second vacuum coupling connected in pneumatic communication with the resin vial; an air inlet coupling connected in pneumatic communication with the resin vial; a first gripping member operationally connected to the fourth second end; a second gripping member operationally connected to the fifth first end; and a rotation fixture connected between the jig and a stationary reference support structure. The jig may be rotated at least about 180 degrees relative to the reference support structure and a workpiece may be interference fit between the first and second gripping members. The first second end is slideably connected into the second first end; the second second end is slideably connected into the third first end; the third second end is slideably connected into the fourth first end; and the fifth second end is connected in pneumatic communication to the first tubular member and positioned near the first first end.

In yet another preferred embodiment, the present invention relates to an adapter for use with a composite socket. The adapter includes a substantially cylindrical ring portion, a connector portion coupled to the ring portion, and at least one resin port extending through the ring portion. The connector portion is adapted to connect to a prosthetic limb. The resin port is adapted to transfer resin from a resin reservoir onto a preform.

In still another preferred embodiment, the present invention relates to a preform for use with a composite socket, including a first set of parallel fibers characterized by a first axis, a second set of parallel fibers characterized by a second axis, and a third set of parallel fibers characterized by a third axis. The first, second and third axes intersect at acute angles relative to each other and are triaxially interwoven into a socket shape conforming to a residual limb shape of a prosthetic wearer.

One object of the present invention is to provide an improved socket. Related objects and advantages of the present invention will be apparent from the following description.

### BRIEF DESCRIPTION OF THE DRAWINGS

FIG. 1 is a schematic view of a first embodiment of a jig for forming a composite socket of the present invention.

FIG. 2 is a schematic view of the resin vial portion of the embodiment of FIG. 1.

FIG. 3A is a side elevation view a lamination dummy.

FIG. 3B is a top plan view of the dummy of FIG. 3A.

FIG. 4 is a partial schematic view of the jig of FIG. 1 including a microprocessor operationally connected to the vacuum and pressure valves.

FIG. 5 is side sectional view of an adapter of a second embodiment of the present invention.

FIG. 6A is a top plan view of an adapter of a third embodiment of the present invention.

FIG. 6B is a side sectional view of the adapter of FIG. 6A.

FIG. 7A is a top plan view of an adapter of a fourth embodiment of the present invention.

FIG. 7B is a side sectional view of the adapter of FIG. 7A.

FIG. 8A is a top plan view of an adapter of a fifth embodiment of the present invention.

FIG. 8B is a side sectional view of the adapter of FIG. 8A.

FIG. 9 is a view of the adapter of FIG. 8B including a first adapting receiver inserted thereinto.

FIG. 10 is a view of the adapter of FIG. 8B including a first lamination dummy inserted thereinto.

FIG. 11 is a top schematic view of a fiber preform woven with an adapter according to a sixth embodiment of the present invention.

FIG. 12A is a top plan view of an adapter of a seventh embodiment of the present invention.

FIG. 12B is a side sectional view of the adapter of FIG. 12A

FIG. 13 is a side schematic view of the embodiment of FIG. 11 including a lamination dummy 18 in place over the mold.

FIG. 14A is a first perspective view of a braider of an eighth embodiment of the present invention.

FIG. 14B is a partial perspective view of the braider of FIG. 14A.

FIG. 14C is a partial side elevation view of the braider of FIG. 14C.

FIG. 14D is a schematic illustration of the braider of FIG. 14A.

FIG. 15A is a side elevation view of a biaxially woven preform produced on the braider of FIG. 14A.

FIG. 15B is a side elevation view of a triaxially woven preform produced on the braider of FIG. 14A.

FIG. 16A is an enlarged partial side elevation view of the preform of FIG. 15B showing the triaxial weave pattern.

FIG. 16B is a schematic view of a triaxial weave pattern.

FIG. 17 is a schematic view of a biaxial weave pattern.

FIG. 18 is a partial cut-away perspective view of a socket of a ninth embodiment of the present invention.

FIG. 19 is a diagramatic view of the method of using the jig of FIG. 1.

### DESCRIPTION OF THE PREFERRED MODE OF THE INVENTION

For the purposes of promoting an understanding of the principles of the invention and presenting its currently understood best mode of operation, reference will now be made to the embodiments illustrated in the drawings and specific language will be used to describe the same. It will nevertheless be understood that no limitation of the scope of the invention is thereby intended, with such alterations and further modifications in the illustrated device and such further applications of the principles of the invention as illustrated therein being contemplated as would normally occur to one skilled in the art to which the invention relates.

It is well known in the art that prosthetic limbs are connected to their wearers by means of a socket worn over the end of the residual limb and including an adapter for coupling to the prosthetic limb. Most, if not all, sockets are custom built to fit their wearers, since the configuration of the residual limb at the interface is unique to the wearer's circumstance. The fit of the socket to the residual limb is crucial to the performance and comfort of the prosthetic limb. It is at the limb/socket interface that the forces and stresses are transferred from the limb to the wearer, and so a poor interface results in painful stress concentrations and distributions for the wearer. A bad interface means a bad limb.

The socket includes two major components, the composite material and the adapter. As described above, adapters come in three general designs, flat plate with standardized metric four hole square pattern, three-winged threaded hole with clamp, and an inverted tetrahedron with a dome and four wings. Each design includes large “wings” to prevent the adapter from being torn out of the socket during service. These adapters are typically formed from a structural material, such as stainless steel or titanium.

The composite material includes a fiber-based textile preform that is conformed to the shape of the wearer's residual limb and a matrix material that is applied to the preform to fill the spaces between the fibers to provide rigidity, to interconnect the fibers, and to distribute stresses more isotropically. The matrix material is typically a thermoplastic or thermosetting resin, although any convenient matrix materials that may be applied in liquid form to substantially wet the fibers and harden therearound may be selected. The preform is preferably formed of carbon fiber, fiberglass, thermoplastic fibers or the like, but may include any convenient fibers that are characterized by the high strength and stiffness properties and are wetably compatible with the selected resin material, as well as fibers suited for cosmetic surface finishes or “beauty coats”, such as nylon, acrylics, and the like. The preform is preferably biaxially woven and more preferably triaxially woven (such as is described in U.S. Pat. No. 3,446,251, issued to Dow on May 27, 1969 and incorporated herein in its entirety), such that forces acting on the socket made from the preform are more evenly distributed.

The composite is formed by hand-controlled vacuum-assisted resin injection into the preform, where the resin flows to substantially permeate the preform and fill in the gaps between the fibers. Resin injection may be performed by hand pressure and stringing to force resin into the lay-up, such that resin is advanced through the fiber preform by vacuum and stringing process, or may be accomplished mechanically, and preferably automatically.

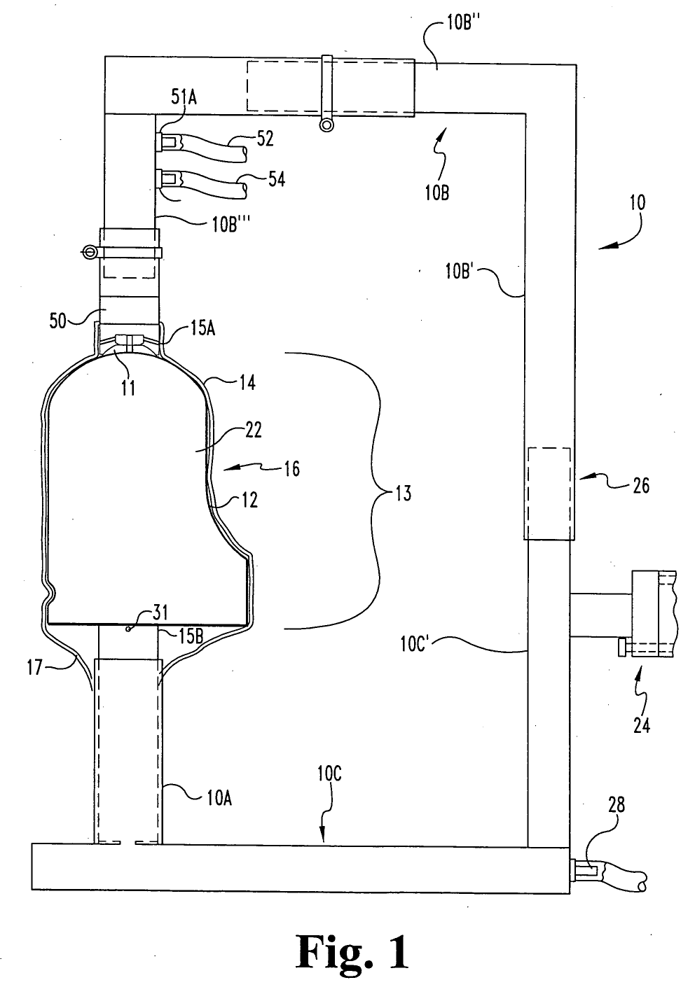

FIG. 1 illustrates one embodiment of the present invention, a system 5 for fabricating prosthetic sockets. The system 5 includes a laminating/alignment fixture 10, or jig. The jig 10 includes rotation fixture 24 attached thereto, such that the jig 10 is rotatable through at least 180 degrees such that transfers of prosthetic alignment can be performed and the partially completed socket assembly can be inverted for gravity assisted laminating. The jig 10 includes several degrees of freedom, which are indexed such that multiple transfers can be accomplished and recorded. The jig 10 is slideable up and down via a telescoping connection 26 to lengthen or shorten the distance between the upper and lower attachment points. The jig 10 is also fully adjustable to allow alignment of the upper and lower attachment points.

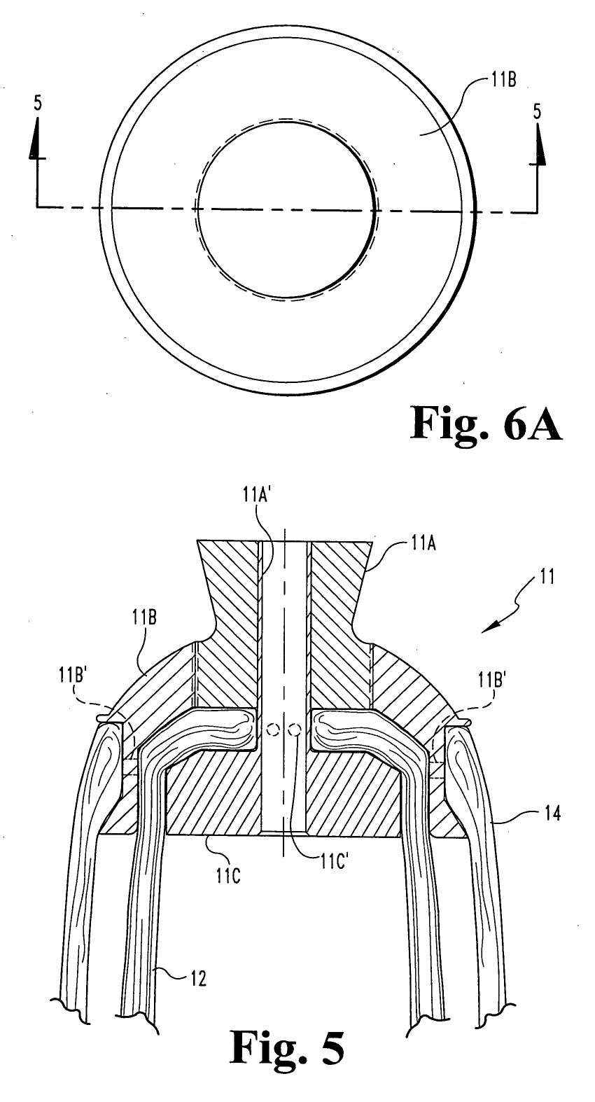

The alignment jig 10 is preferably fabricated from hollow-square or round stock aluminum or stainless steel to allow for vacuum and pressure to be channeled through the fixture and attachment points. A vacuum fitting 28 is provided in the lower section of the jig for feeding the lower attachment point via vacuum hose 29, which is connected to timer/valve system 30 (see FIG. 4). A piece of detachable round tubing 10a defines the first vacuum path and holds the mandrel 31 of the plaster model. The top section 10b and lower section 10c of the jig are hermetically sealed to create the vacuum/pressure path at the other end of the jig. Tubing 10a is a part of lower section 10c. Upper section 10b comprises vertical portion 10b′, horizontal portion 10b″ and vertical sections 10b′″ and 10d leading to a resin vial 50 mechanically and pneumatically connected thereto. In general, all tube sections are slideably connected to each other, such that for any given connection of tubes, one tube end is sized to slide into the other for a predetermined distance. Section 10b′″ further includes pneumatic connection ports 51a and 51b, to which positive pressure 52 and vacuum 54 hoses may be attached. The ports 51a and 51b are in pneumatic communication through section 10b′″ and 10d with the resin vial 50. Section 10a extends from section 10c and terminates in vacuum port 31. Vacuum port 31 is therefore in pneumatic communication through sections 10a and 10c with vacuum fitting 28.

The jig is adapted to hold a workpiece 13 between sections 10a and 10d′″, and more specifically between the gripping connector 15a positioned over resin vial 50 gripping connector 15b, slideably connected to member 10a and including vacuum port 31. The workpiece may comprise a mold 22 over which a preform 16/adapter 11 combination has been positioned. As will be discussed in further detail below, this configuration allows the amount of pressure applied to the workpiece 13 to be varied during the lamination process to inject and then withdraw resin in the lay-up.

The workpiece 13 includes a mold 22, which is a positive three-dimensional facsimile of the wearer's residual limb upon which a prosthetic is desired to be worn. The mold 22 is covered by a preform 16, which is preferably shaped to form-fittingly cover the mold 22. The preform 16 includes at least one, and preferably multiple, layers of criss-crossing fibers. The preform may include layers of biaxially woven fibers (with each layer preferably having a different axial orientation than the ones below and above it), or, more preferably, layers of triaxially woven fibers. In some embodiments, layers of other materials, both fibrous and otherwise, may be interspersed with the woven layers to tailor the properties of the preform as desired. The fibers may also be provided as braided strands, which are in turn woven as discussed above, into layers.

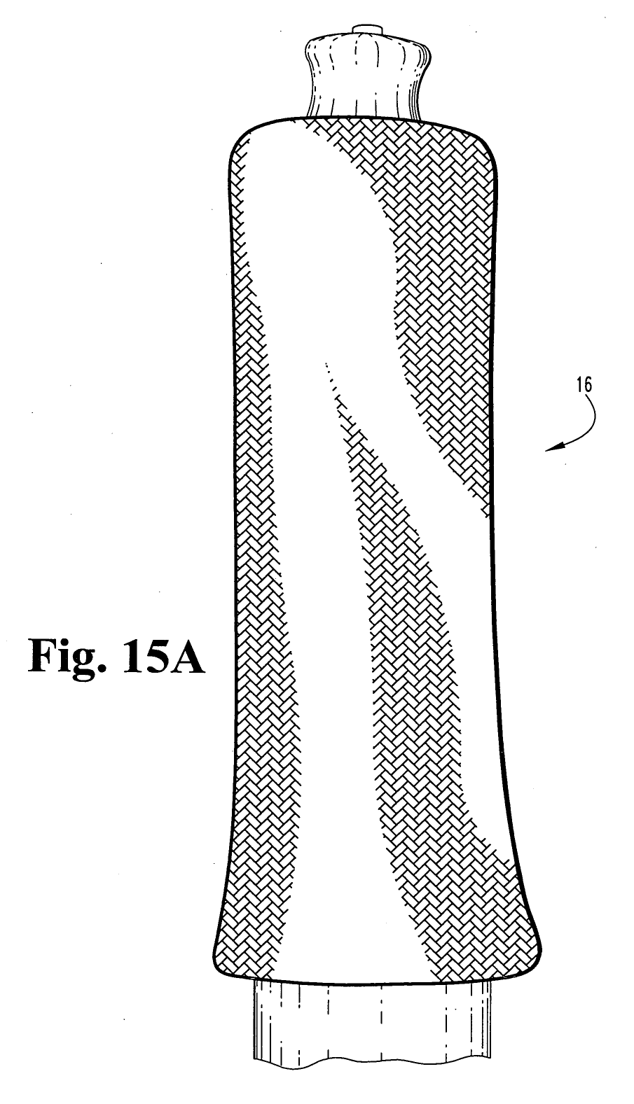

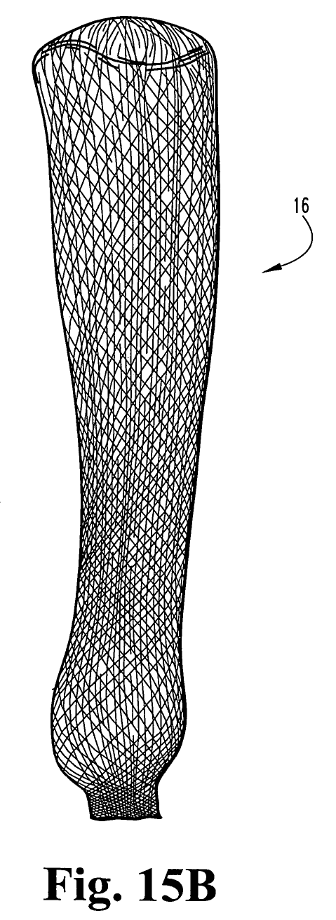

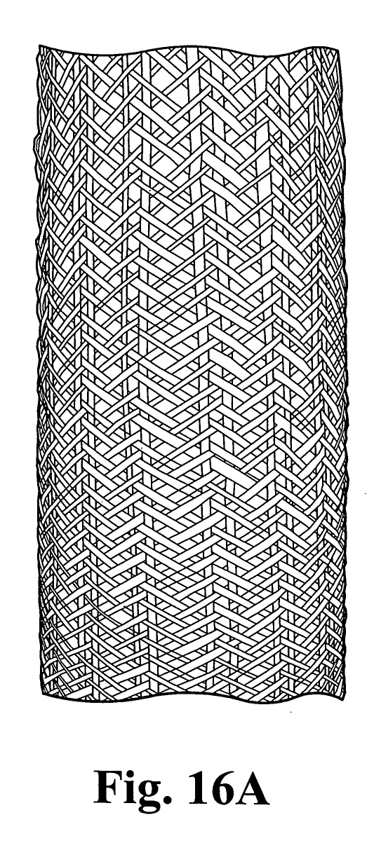

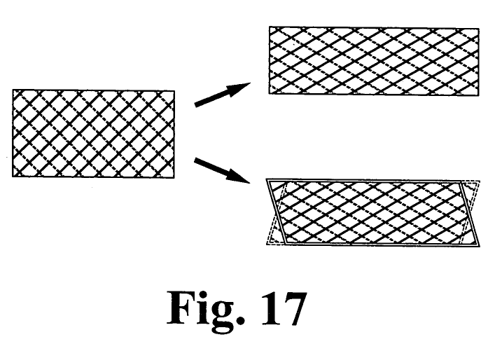

FIGS. 14A-14D illustrate a preferred embodiment weaving apparatus or braider 100. The braider 100 includes a plurality of spools 110. Each spool is wound with fibers 115, which are woven in a predetermined pattern onto a mandrel 120. The mandrel 120 may be a dummy cylinder or may be a mold 22 having the shape of the wearer's residual limb. The result is either a generally cylindrical preform 16 (see FIG. 15A) or a preform 16 having a shape conforming to the wearer's residual limb (see FIG. 15B). It is important to note that a generally cylindrical preform 16 is typically woven using a biaxial fiber weaving process, since a biaxially woven preform 16 may be distorted by bulging and/or stretching relative to its central axis. Triaxially woven preforms are generally limited to weaving directly to shape, such as over a positive mold 22, since triaxially woven material may be only slightly distorted, if at all, by the application of external forces. FIGS. 16 A and B illustrate typical triaxial weave patterns, while distorted and undistorted biaxial weave patterns may be seen in FIGS. 15A and 17.

One aspect of the invention includes an automated fiber-preforming filament winding process. Layers of fibers, each having a predetermined predominant direction of fiber orientation, are built up over the mold or mandrel layer-by-layer until the proper thickness and orientation are completed. A preform capable of being easily pulled over a mold after its manufacture is the result. The instant invention includes the process and the preform made therefrom.

Although the total amount of fiber may be fixed for a given socket size, the stacking of the fiber in the third axis through the thickness is very important to the quasi-anisotropic strength characteristics of the resultant preform. By overlapping fiber layers, varying thickness preforms can be manufactured while still maintaining the proper amount of permeability per cross-sectional area.

The present invention includes the process of wrapping a band of filaments around a rotating mandrel. A horizontally moving carriage is oriented perpendicularly to a long axis of the mandrel, with a feed eye, or roller, delivering the fiber band to the mandrel. The fiber band is continuous except for the two cut ends of the band. The fiber over the adapter is tied and wrapped over the exterior of the part maintaining a grip on the part until laminating. There will also be an inner layer of fiber that covers the under side of each of the three adapters.

Another aspect of the invention includes triaxial and biaxial filament braiding to create two-dimensional composites of varying thickness while maintaining the desired fiber orientation. Many bands of fiber are fed into the plurality of feed eyes at once. As few as ten and as many as 800 spools of fiber may be fed at once depending on the diameter of the braided sleeve desired. As the fiber bundles are fed, they are crossed over and under each other creating a woven tubing. Approximately half of the fiber carriers are rotating clockwise and the other half are rotating counterclockwise. Additionally, a third set of fibers can be applied in the axial or “machine” direction, i.e. along the major axis of the mold.

Horn gears hold the carriers and rotate passing the fiber bands over and under as they pass one another. The process yields low angle preforms that accomplish through the thickness stacking at the small diameter and thinner distributions at the larger diameter while still maintaining coverage. The preforms may be braided onto the adapter or tied on after the braiding process. Alternately, they may be braided directly over the custom socket mold integrating the adapter during the process.

It should be understood that in either of our new methods, several layers of differing orientation angles between 0 and about 85 degrees relative to the major axis of the prosthesis/limb, such as 45, 30 and 15 degrees, may be produced and incrementally disposed on the work piece to optimize a single preform for a specific type of socket, such as below-knee or above-knee amputation site.

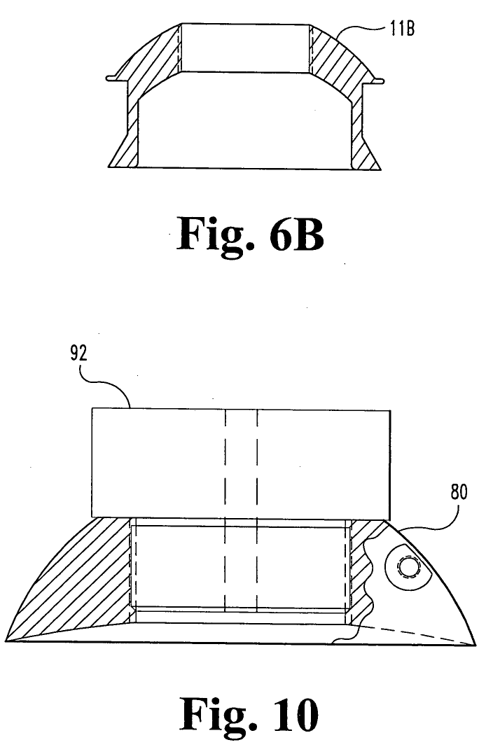

FIGS. 5-10 and 12A and B illustrate various adapter 11 designs, each to be discussed separately and in greater detail below. One common characteristic of the adapters 11 of the present invention is the presence of resin ports extending therethrough for the transport of resin from the resin vial 50, through a dummy 18/92 (if one is included), through the adapter 11, and into the preform 16. In some embodiments, the vial 50 may inherently include a dummy feature.

Preferably, the adapters 11 are formed of structural materials, and more preferably the adapters are formed of titanium, stainless steel and/or aluminum to create a region of increased toughness positioned between layers of preform 16 (and, eventually, the composite socket 130). In other words, the composite benefits from the anisotropic strength/modulus of elasticity of the metal layer while enjoying the more isotropic characteristics of the adjacent composite layers. As detailed below, the adapter 11 may be of any convenient design allowing for the ready application of resin to the preform 16 to make the composite socket. The combination of resin permeable adapter 11 and woven preform 16 based composites yield lighter and more readily adjustable modular limb systems. The socket preform/adapter design integrates resin flow ports in the adapter 11 to facilitate resin flow and wet-out as well. In one aspect of the invention, fiber placement and resin infusion is automated.

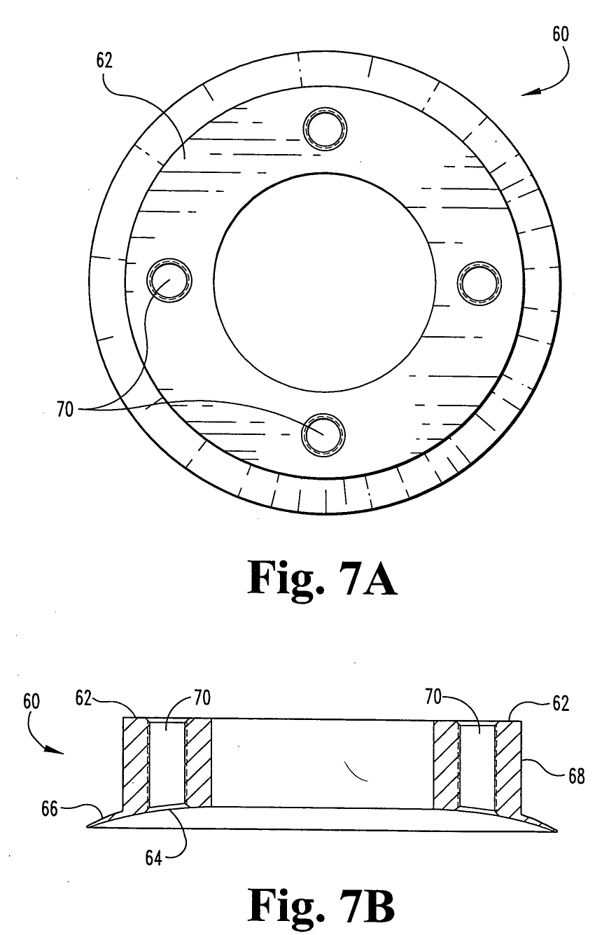

Except for its attachment surface, the metal adapter 11 as shown in FIG. 5 is positioned between the internal layer 12 and external layer 14 of composite, which is now defined by the inner fiber resin preform. The adapter 11 as shown in FIG. 5 has a tetrahedral insert 11a that is threadably received or by dress-fitting within a dome housing 11b (FIGS. 6A and 6B). The insert 11a is preferably formed from titanium or stainless steel, but may alternately be formed from any convenient structural material. The tetrahedron 11a and its dome 11b are exposed as the attachment point of the other prosthetic components, such as tube clamp adapters and double pyramid adapters. Adapter 11 also includes an internal plug or insert 11c located within the concave portion of adapter 11 and that extends axially through the central bore 11a′ of the tetrahedral insert 11a. Insert 11c has ports 11c′ to introduce resin into the inner preform 12. As shown in FIG. 5, adapter 11 is provided with resin ports 11b′ to allow resin to reach outer preform 14. FIGS. 6A and 6B present a top plan view and cross-section, respectively, of the dome housing 11b in FIG. 5 without the inserts 11a or 11c arranged therein.

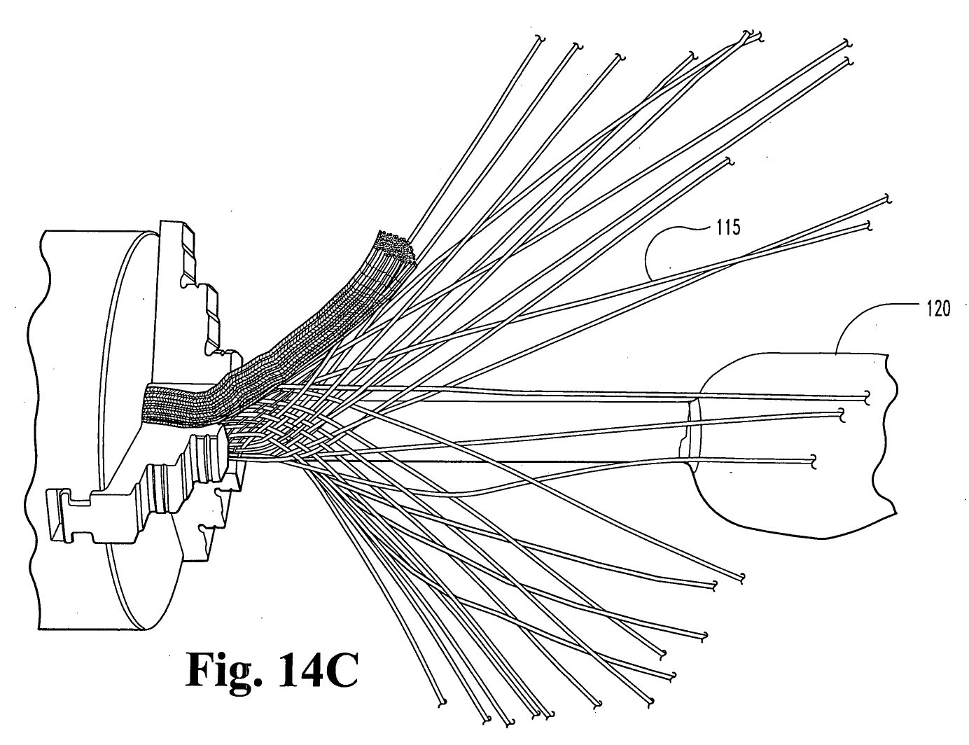

The inner insert 11c is shown in isolation in FIGS. 12A and 12B, comprising an axial portion 40 that extends through the central bore 11a′ of tetrahedral insert 11a (FIG. 5), an annual base portion 42 including a vertical outer wall 42a and a horizontal surface 42b, and a beveled portion 42c connecting outer wall 42a and horizontal surface 42b. While a currently preferred cross-sectional configuration of insert 11c is shown in FIG. 12B, it should be understood that that specific configuration is not critical to the effective operation of this invention. In the currently preferred embodiment, the cross-sectional configuration of insert 11c correspondences to the interior portion of adapter dome housing 11b shown in FIG. 6B. Again, the function of insert 11c is to secure or sandwich the inner and outer layers of the fiber preform at the distal end of the prosthetic. Alternatively, the cross-configuration of insert 11c, as well as of adapter housing 11b, can take a different shape, such as curved or spherical.

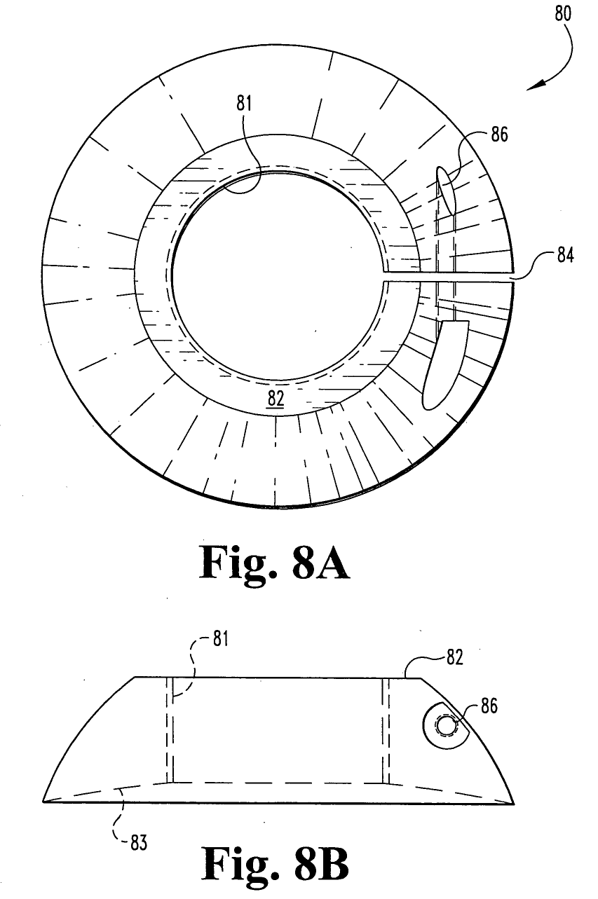

FIGS. 7A and 7B show a four-hole adapter 60, preferably formed from aluminum. The flat surface 62 of the adapter is exposed for attachment of four-hole rotatable adapters, four-hole knees, four-hole pyramid adapters or the like. Adapter 60 has an inner curved surface 64, outer curved surface 66 and outer vertical portion 68 of the adapter are also reinforced with the socket composites. The outer curved surface 66 preferably has a radius slightly smaller than that of curved surface 64. Adapter 60 is preferably provided with four longitudinal through-holes 70 forming a standard four-hole square pattern, commonly referred to in the trade as an “European Four-Hole Pattern.”

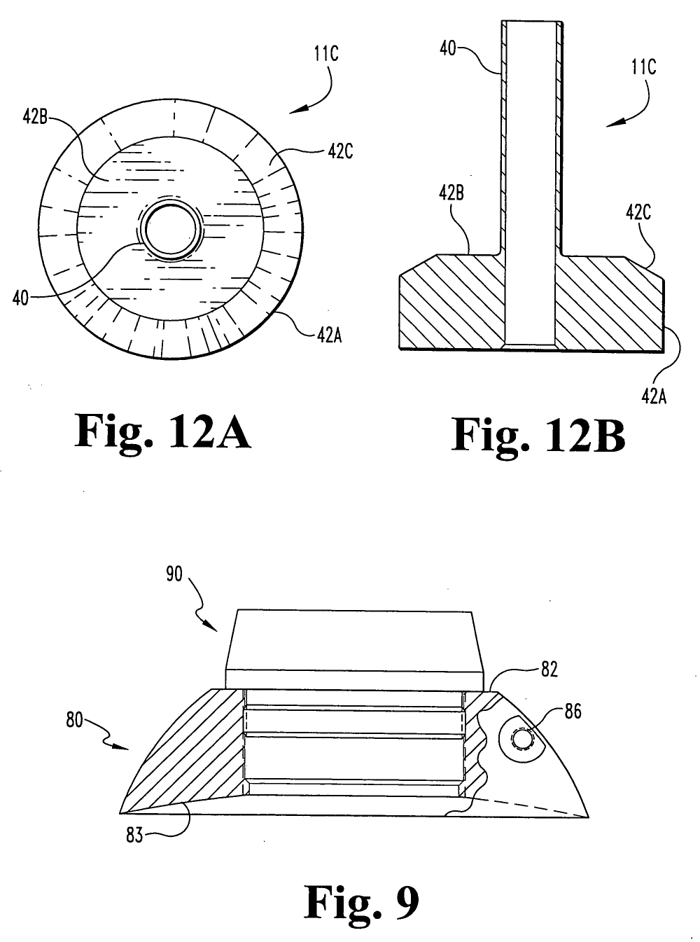

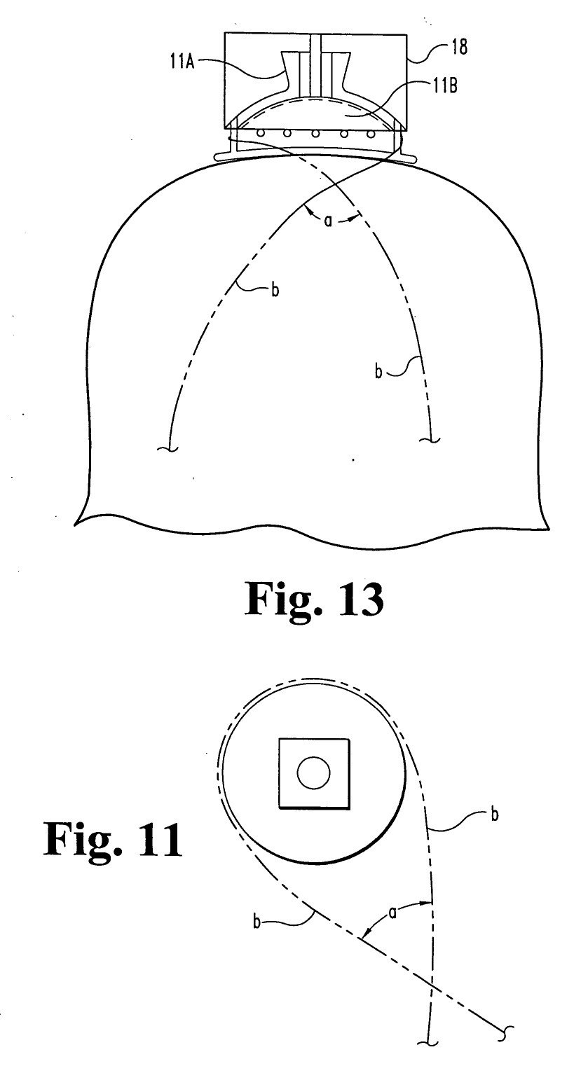

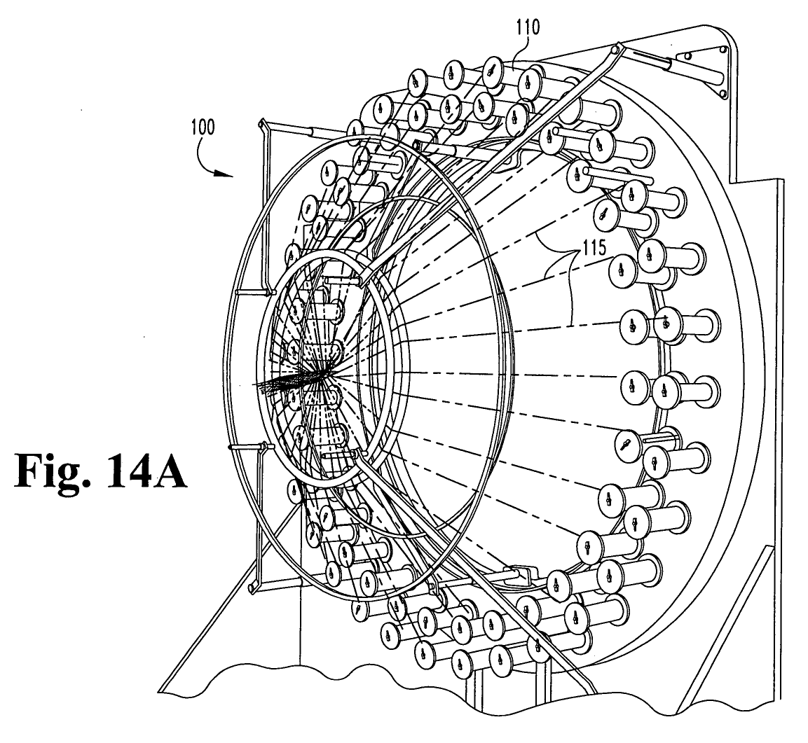

Another aspect of the invention shown in FIGS. 8 and 9 provides a large-hole rotatable adapter 80 having a central opening 81 and an exposed horizontal surface 82 serving as a stop for a threaded pyramid receiver or threaded pyramid adapter known in the art. This adapter 80 is preferably formed from aluminum and has a slot 84 cut from the outer edge to the central opening 81. There is a threaded hole 86 for receiving a tightening screw disposed perpendicular to the slot 84 that extends across the slot to decrease the diameter of central opening 81, thereby facilitating a prevailing torque on the threads of the adapter 80. This also provides a rotational adjustment to the long axis of the prosthesis. Adapter 80 is also preferably provided with a slightly concave lower surface 83. As shown in FIG. 9, adapter 80 is intended to frictionally or threadably receive in central bore 81 a pyramid adapter receiver 90, an inverted tetrahedral insert (such as insert 11a), or the like, each of which is known in the art. FIG. 10 shows large-hole adapter 80 coupled with a lamination dummy 92 to prevent the large counter bore 81 from filling with resin during the forming process. Flat surface 82 remains free of resin and composite during the infiltration process.

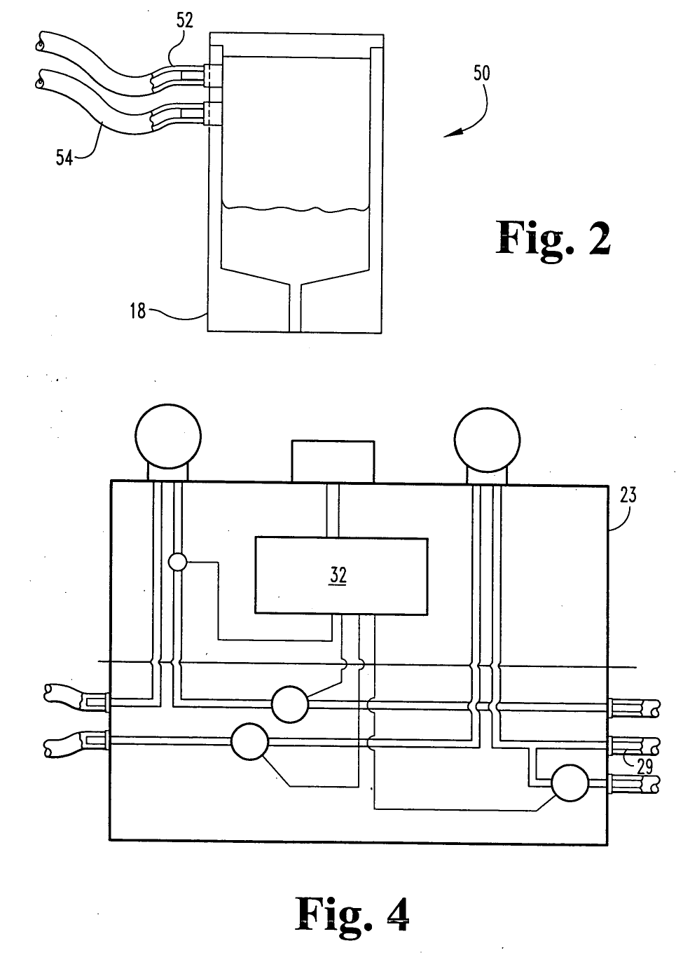

An alignment/laminating fixture 10 shown in FIG. 1 is used to perform laminating tasks and prosthetic alignment transfers. Since there is a concentration of thickness around the adapter 11 (above and beneath it), a slight pressure of 10-15 psi is applied to the resin vial 50 to actuate flow of resin from the vial 50 through the adapter 11 and onto the preform 16 until the inner portion 12 and outer portion 14 of the preform 16 are wetted out substantially evenly permeated with resin. This is accomplished by a positioning a reusable rigid vial 50 (FIG. 2) that has a regulator and a three-way (or an automated valve system) stopcock (not shown) above the reservoir. In another embodiment, an automated valve system may be used instead of a manual stopcock. In yet another embodiment, a mechanical ram may be used to urge the flow of the resin.

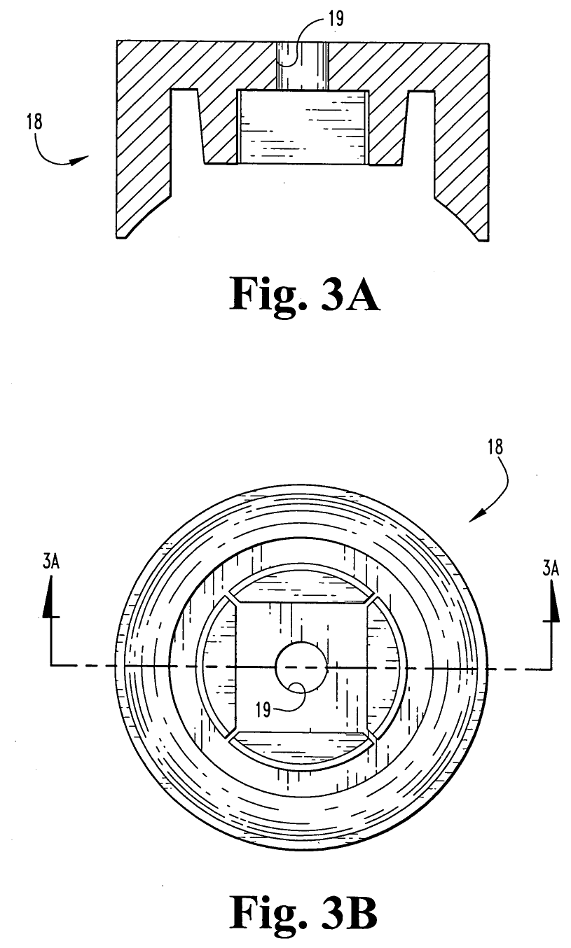

In process, the resin vial 50 is clamped over a plastic lamination dummy 18 with resin injection ports feeding the ports in the adapter 11. In a preferred embodiment, dummy 18 has a central axis port 19 shown in FIGS. 3A and 3B. The lamination dummy may also be provided with additional injection ports to direct resin as desired. Alternatively, the vial may be detachably secured to the adapter 11 by itself. The dummy or vial is locked onto the adapter 11 with screws or a clamp. The flexible bagging film (PVA bag 17) receives resin, which permits the adapter assembly to be lifted off the mold enough to allow efficient wet-out. The proximal section of the mold is preferably under vacuum. A high degree of vacuum is preferred. Favorable results have been obtained using a vacuum of between about 20 to 29 inches of mercury (in Hg).

After the preform 16 is completely wetted or infiltrated with resin, the positive pressure is switched to vacuum, which pulls excess resin back into the vial 50 and into the bleeder (not shown) at the proximal end of the lay-up (i.e., the distal end of the socket 130). In this context, the bleeder is a wad of porous material intended to absorb excess resin to prevent excess resin from filling up and clogging the first vacuum path. One embodiment is essentially manually actuated with the apparatus operably connected to a vacuum pump. A preferred embodiment employs an automated method, which is illustrated diagrammatically in FIG. 4.

With reference to FIG. 4, a timer/valve system 30 is used to automate resin infiltration and debulking. In a preferred embodiment, two timer algorithms are run in the microprocessor 32; one timer algorithm runs the vacuum pump and automatically turns it off after a predetermined period of time coinciding with when the resin is cured, and the second timer algorithm runs pressure to the vial 50 and switches to vacuum after infiltration is complete. The second vacuum debulks the lower half of the fiber preform 16 by returning excess resin to the vial. The vacuum source at the top of the mold 22 (which is inverted) continues drawing resin in the gravity direction until the viscosity of the resin increases past a predetermined threshold value (i.e., gels or otherwise solidifies). Both vacuum sources are preferably fed from the same pump. All timing and switching is automated within the microprocessor 32 so the normal process of stringing resin is eliminated. A manual override system, however, allows emergency control of the pressure/vacuum switching.

In practice, the fiber preform 16 is pulled over the mold 22 after application of a PVA bag parting film 17 thereto. The adapter 11 is locked to section 10b of the jig 10, maintaining its orientation after the transfer process has been completed. A second PVA bag 17 is then pulled over the mold with the preform 16 already applied and sealed creating the first vacuum path. The resin vial 50 is attached to the adapter via lamination dummy 18 or directly to the adapter 11.

The mixed resin is poured into the vial 50 and the vial is closed and sealed by an O-ring, clamp or tape. The stopcock is manually opened, allowing the resin to enter the lay-up through resin ports in the vial, in the dummy (port 19 in FIG. 3A) (if used) and in the adapter 11. A positive pressure of between about 5-15 psi is applied to the resin chamber either directly or indirectly through a ram or bladder, which substantially prevents the air bubbles from forming in the resin. Switching the pressure and vacuum on and off is accomplished manually or automatically using a computer algorithm or a timer. (It should be noted that automatic switching is preferred as more accurate.) The system 30 may deliver pressure or create a vacuum via hoses 52 and 54, respectively. Hoses 52 and 54 may be attached directly to vial 50 as shown in FIG. 2, or attached to the jig 10 as shown in FIG. 1.

The automated embodiment provided by this invention includes solenoid or mechanical valving controlled by a microprocessor 32 (FIG. 4). Timing and valving is controlled from the microprocessor 32 as well. Preset timing and switching is performed based on predetermined data corresponding to each adapter/preform size. A manual override is preferably included for emergency situations. After the positive pressure cycle has run, the second vacuum path is switched to vacuum to withdraw excess resin from the lay-up. Once the resin is cured, the vacuum pump and compressor are shut down. A preferred embodiment includes a manifold operably connected to the system for performing more than one operation simultaneously or from a central location in a prosthetic facility.

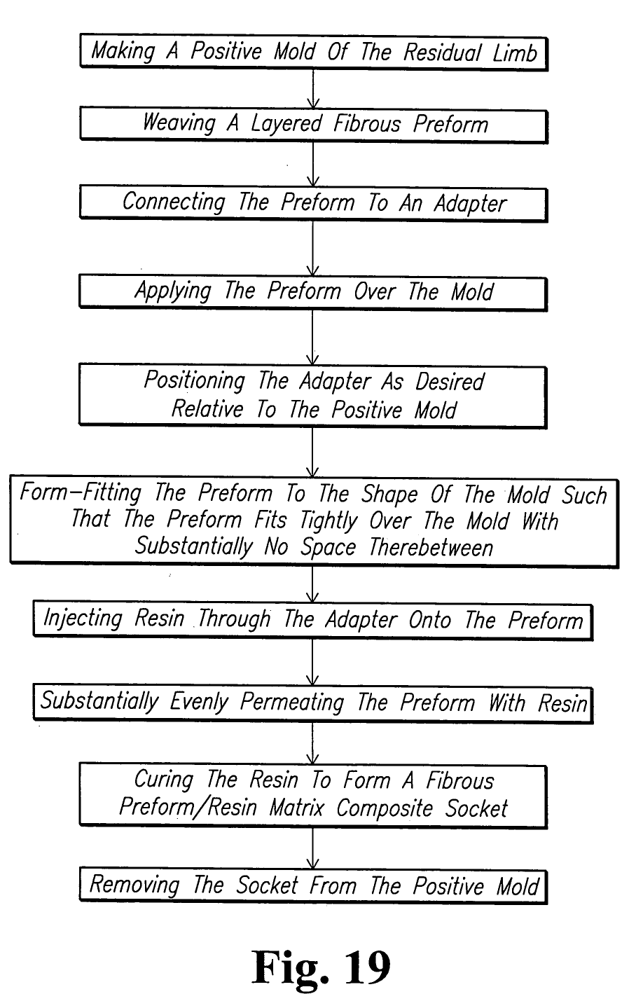

The computer automated lamination cycle will now be described as illustrated in FIG. 19. Once the unit is powered on, the user is prompted to select a program or manual mode. The vacuum pump (shown schematically in FIG. 4) is powered up shortly after the program is selected and will hold until the first PVA bag, adapter/preform and second PVA bag are applied. Next, the resin is introduced into the vial 50 which is then attached to the resin inlet. Alternatively, the vial can be filled with resin while detached and then the filled vial 50 may be attached to the lamination dummy or, in some instances, directly to the adapter 11, 60 or 80. Vial 50 should be formed from a non-stick substance so that the laminating resin will not adhere to it. Nylon 66 or a like thermoplastic is preferred. The pressure/vacuum program is then actuated. System 30 (shown in FIG. 4) is preferably provided with a keypad interface or connected to an external computer in order for the user to operate and/or program the system. As an initial step, pressure is applied for a predetermined time. If there is a significant drop in pressure, an alarm will sound, and the positive pressure will be interrupted. A leak in the PVA bag or an exhaustion of resin supply are examples that may trigger such a pressure interruption. When the resin vial 50 is near empty, the positive pressure is deactivated. In such a case, the preform will not yet be completely wetted and, thus, the resin vial will pause (i.e. the positive pressure will temporarily stop), allowing the remaining portion of the preform to be infiltrated by the resin. The mold and preform may be inverted if desired so that gravity can facilitate resin infiltration.

Next, the vacuum line to the vial 50 is actuated. The excess resin is returned to the vial at the distal end and taken up in the bleeder at the proximal end under the mold at the top of the lay-up (the bleeder is under the mold at the “top” of the lay-up if the mold has been inverted). The vacuum pump continues to operate until the resin has gelled and set. Once the resin has gelled and set, the vacuum pump is deactivated. The user can then power down the unit or select another program.

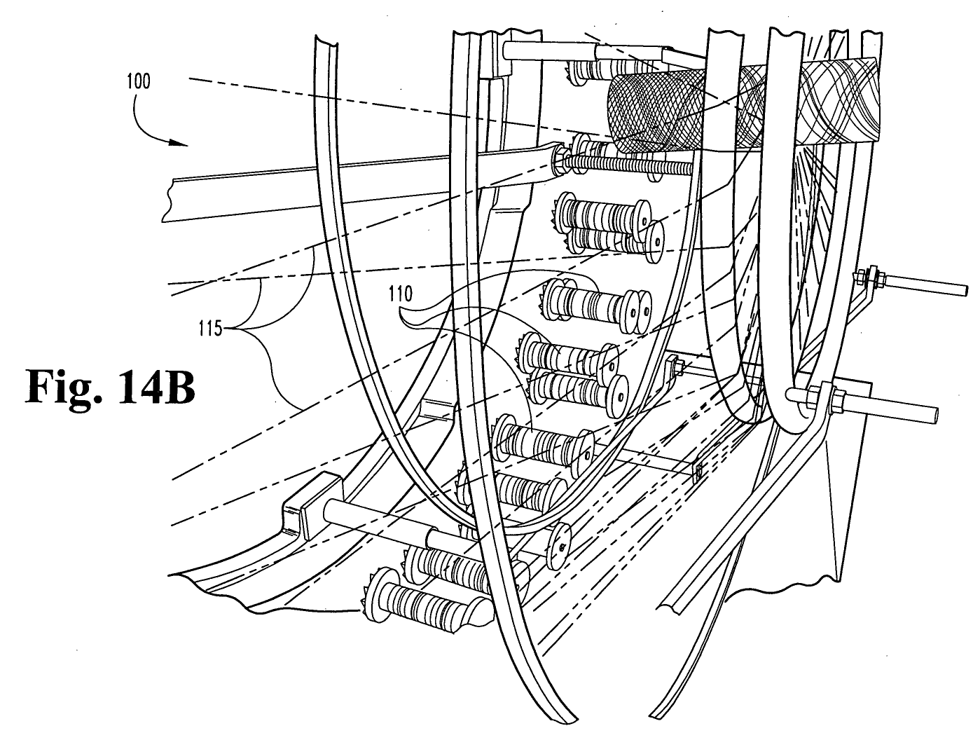

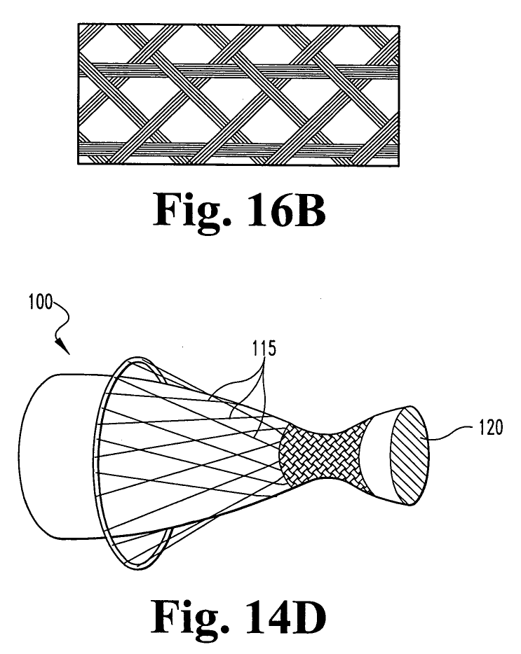

FIGS. 11 and 13 show the fiber preform 16 woven with an adapter 11 and lamination dummy 18 in place over the mold. In a preferred mode the fiber is wound along a path “b” about a mandrel onto an adapter, such as adapter 11, 60 and 80 of this invention (adapter 11 is depicted in FIG. 13), after which the mandrel is removed creating a dry preform/adapter assembly. The fiber may be wound, woven or braided. If wound or woven, the fiber path “b” may cross over itself at an included angle “a” between about 15 to 45 degrees, preferably about 20 degrees, while it's not necessary that the fiber paths cross at all. As shown in FIG. 13, the adapter is preferably equipped with resin injection ports that extend radially through the adapter, such as ports 11b′ as shown in FIG. 5.

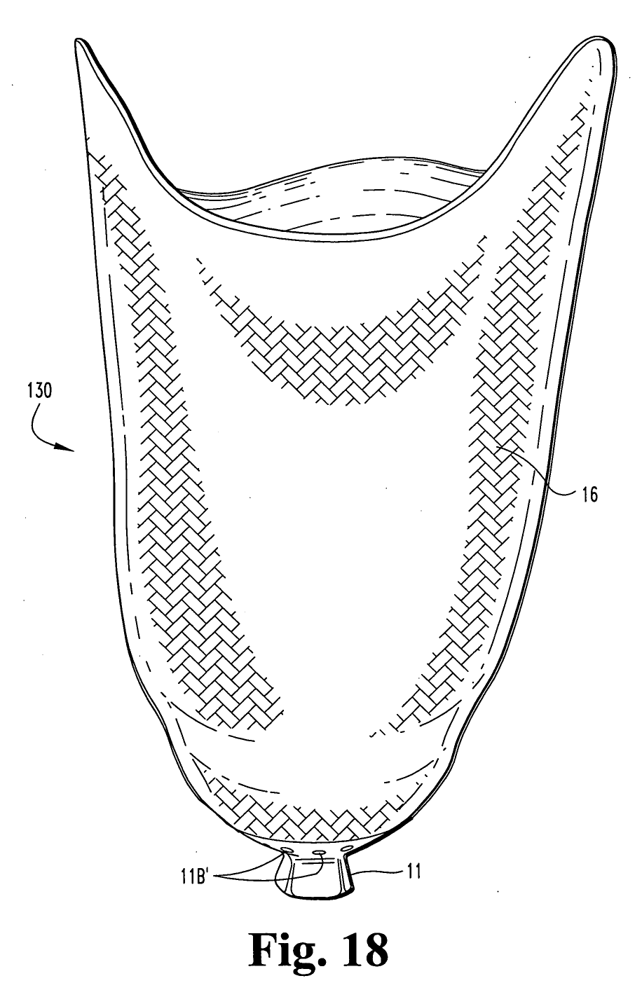

FIG. 18 illustrates one embodiment of a socket 130 made according to the above-described method. The socket 130 is generally conical and includes an adapter 11 protruding from an area generally on or near its apex. The adapter 11 includes a plurality of resin ports 11b′ formed therethrough through which resin was flowed onto the now-encased preform 16.

While the invention has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nearly infinite number of insubstantial changes and modifications to the above-described embodiments and that it would be impractical to attempt to describe all such embodiment variations in the present specification. Accordingly, it is understood that all changes and modifications that come within the spirit of the invention are desired to be protected.

I claim:

1. A method for making a socket to be worn over a residual limb for connection of a prosthetic limb thereto, comprising the steps:
a) making a positive mold of the residual limb;
b) weaving a layered fibrous preform;
c) connecting the preform to an adapter;
d) applying the preform over the mold;
e) positioning the adapter as desired relative to the positive mold;
f) form-fitting the preform to the shape of the mold such that the preform fits tightly over the mold with substantially no space therebetween;
g) injecting resin through the adapter onto the preform;
h) substantially evenly permeating the preform with resin;
i) curing the resin to form a fibrous preform/resin matrix composite socket; and
j) removing the socket from the positive mold;
wherein at least one layer of the preform includes criss-crossing fibers oriented in at least two axial directions.

2. The method of claim 1 further comprising the step of:
k) before step f, tightly stretching a bag parting film over the positive mold.

3. The method of claim 2 further comprising the steps of:
l) after step f and before step g, positioning a substantially airtight bag over the preform to define a first space between the bag parting film and the substantially airtight bag; and
m) after step l and before step g, reducing the air pressure in the first space.

4. The method of claim 3 further comprising the step of after step h and before step i, applying negative pressure to the first space to withdraw excess resin.

5. The method of claim 1 wherein the layered preform is triaxially woven onto the positive mold.

6. The method of claim 1 wherein the layered preform includes at least two biaxially woven layers.

7. The method of claim 6 wherein the at least two biaxially woven layers are each characterized by a pair of fiber axes and wherein the no axis is codirectionally oriented with the other three axes.

8. The method of claim 6 wherein the layered preform includes at least one uniaxially oriented layer positioned between two biaxially woven layers.

9. A socket to be worn over a residual limb for connection of a prosthetic limb thereto, comprising:
an adapter having at least one resin port formed therethrough; and
a composite shell having an inner surface and an outer surface and connected to the adapter
wherein the composite shell extends generally cylindrically away from the adapter;
wherein the inner surface of the socket is custom-molded to conform to the contours of the residual limb of a desired wearer;
wherein the adapter protrudes through the outer surface of the composite shell; and
wherein the composite shell further comprises:
a cured resin matrix; and
a woven fiber preform embedded in the resin matrix.

10. The socket of claim 9 wherein the fiber preform is triaxially woven.

11. The socket of claim 9 wherein the fiber preform further comprises:
a first biaxially woven layer characterized by fiber axes oriented in first and second axial directions; and
a second biaxially woven layer characterized by fiber axes oriented in third and fourth axial directions;
wherein the first, second, third and fourth directions are all unique.

12. The socket of claim 10 further comprising a third layer positioned between the first and second layers and wherein the third layer is non-biaxially woven.

13. The socket of claim 10 further comprising a third layer positioned between the first and second layers and wherein the third layer is non-woven.

14. A socket for connecting a prosthetic limb to the residual limb of a amputee, comprising:
an adapter having at least one resin port extending therethrough; and
a generally conical composite material extending from the adapter;
wherein the composite material further comprises:
a resin matrix; and
a multilayer triaxially woven preform embedded in the matrix;
wherein the preform is woven directly onto a positive mold of the residual limb;
wherein the adapter is positioned generally at the apex of the generally conical composite material; and
wherein the adapter protrudes through the composite material.

15. An adapter for use with a composite socket, comprising:
a substantially cylindrical ring portion;
a connector portion coupled to the ring portion; and
at least one resin port extending through the ring portion;
wherein the connector portion is adapted to connect to a prosthetic limb;
wherein the resin port is adapted to transfer resin from a resin reservoir onto a preform.

16. The adapter of claim 15 further comprising a connecting flange portion coupled to the ring portion, wherein the connecting flange portion is adapted to receive a preform.

17. A jig for producing a composite residual limb socket, comprising in combination:
a first substantially L-shaped hollow tubular member having a first first end and a first second end;
a second substantially L-shaped hollow tubular member having a second first end and a second second end;
a third substantially L-shaped hollow tubular member having a third first end and a third second end;
a fourth hollow tubular member having a fourth first end and a fourth second end;
a fifth hollow tubular member having a fifth first end and a fifth second end;
a resin vial connected in pneumatic communication with the fourth second end;
a vacuum port connected in pneumatic communication with the fifth first end;
a first vacuum coupling connected in pneumatic communication with the vacuum port;
a second vacuum coupling connected in pneumatic communication with the resin vial;
an air inlet coupling connected in pneumatic communication with the resin vial;
a first gripping member operationally connected to the fourth second end;
a second gripping member operationally connected to the fifth first end; and
a rotation fixture connected between the jig and a stationary reference support structure;
wherein the jig may be rotated at least about 180 degrees relative to the reference support structure;
wherein a workpiece may be interference fit between the first and second gripping members;
wherein the first second end is slideably connected into the second first end;
wherein the second second end is slideably connected into the third first end;
wherein the third second end is slideably connected into the fourth first end; and
wherein the fifth second end is connected in pneumatic communication to the first tubular member and positioned near the first first end.

18. The jig of claim 17 wherein the second gripping member is slideably connected to the fifth first end and wherein the second gripping member includes the vacuum port.

19. The jig of claim 17 wherein the tubular members are hermetically sealingly connected to each other.

20. The jig of claim 17 further comprising a vacuum source operationally connected to the vacuum couplings and an air source operationally connected to the air inlet coupling.

21. The jig of claim 20 further comprising a microprocessor operationally connected to the vacuum couplings and air inlet, wherein the microprocessor is programmed to selectively actuate and deactuate the vacuum couplings and air inlet at preselected times.

22. A preform for use with a composite socket, comprising:
a first set of parallel fibers characterized by a first axis;
a second set of parallel fibers characterized by a second axis; and
a third set of parallel fibers characterized by a third axis;
wherein the first, second and third axes intersect at acute angles relative to each other;
wherein the first, second, and third sets of fibers are triaxially interwoven; and
wherein the first, second and third sets of fibers are woven into a socket shape conforming to a residual limb shape of a prosthetic wearer.
